(19) United States
(12) Patent Application Publication
(10) Pub. No.: US 2017/0035968 A1
(43) Pub. Date: Feb. 9, 2017

(54) IMPLANTABLE DEVICE FOR AUTOMATIC DELIVERY OF MEDICATION FOR ALLERGIC REACTIONS

(71) Applicants: Alexander Hassan, Ann Arbor, MI (US); Tarek Hassan, Ann Arbor, MI (US)

(72) Inventors: Alexander Hassan, Ann Arbor, MI (US); Tarek Hassan, Ann Arbor, MI (US)

(21) Appl. No.: 15/231,386

(22) Filed: Aug. 8, 2016

Related U.S. Application Data
(60) Provisional application No. 62/202,398, filed on Aug. 7, 2015.

(51) Int. Cl. A61M 5/172; A61K 9/00; A61K 31/137; A61M 5/142

(52) U.S. Cl. CPC A61M 5/1723; A61M 5/14276; A61K 9/0024; A61K 9/0097; A61K 31/137; A61M 2230/20; A61M 2205/50; A61M 2205/3569; A61M 2205/3523; A61M 2205/3379; A61M 2205/8206; A61M 2205/52

(57) ABSTRACT

Apparatus, implanted subcutaneously or in muscle, fat, joint spaces, or body cavities of any type, detects and responds to an allergic and/or anaphylactic reaction. Detection is carried out by monitoring the levels of biomarker molecules that indicate the occurrence of an allergic and/or anaphylactic reaction, such as: histamine, leukotrienes, prostaglandins, cytokines, tryptase, Fc-ε-RI complexes, anaphylatoxin C3a, chymase, carboxypeptidase A, platelet-activating factor⁸, other mast cell degranulation byproducts, or other basophil activation compounds. An appropriate dosage of medication (such as epinephrine, antihistamines, or steroids) is automatically dispensed to mitigate the allergic/anaphylactic response. The simplicity and ease of use of the invention has the potential to save many lives worldwide, while dramatically mitigating the risks of the current methods of handling severe allergic reactions using external auto-injectors. ⁸ Sala Cunill, A., Cardona, V. Biomarkers of anaphylaxis, beyond tryptase. Current Opinion in Allergy and Clinical Immunology. Vol 15(4), August 2015, p 329-336. Doi: 10.1097/ACI.0000000000000184.

### REFERENCE TO RELATED APPLICATION

This application claims priority from U.S. Provisional Patent Applciation Ser. No. 62/202,398, filed Aug. 7, 2015, the entire content of which is incorporated herein by reference.

### FIELD OF THE INVENTION

This invention relates generally to implantable devices and, in particular to a device, system and method for automatic delivery of medication in response to allergic reactions

### BACKGROUND OF THE INVENTION

Approximately 1 in 5 people suffer from allergies in the U.S. today. Many of those affected struggle with severe allergies, for whom an untreated allergic response can lead to anaphylaxis or even a swift death. Sufferers of moderate to severe allergies are instructed to carry a dosage of epinephrine on their person at all times. The most common device for epinephrine distribution is the EpiPen®, of which 50 million units have been distributed over the past 25 years.¹ ¹ https://www.epipen.com/personal-stories

While the EpiPen® auto-injector has been a lifesaving tool for countless cases worldwide, it has several drawbacks. For one, the allergic patient must carry the device with them at all times, which is often impractical or impossible. The epinephrine distribution also does not occur until the patient is suffering from a severe attack. Patients are often hesitant about making the decision to inject until the attack is very serious, which is sometimes too late. During the allergic reaction, the patient must either be physically able to inject himself or herself, or find, ask, and communicate the location of the auto-injector to someone else. During a severe allergic reaction, these conditions are often impossible.

An automatic medication distribution system inside the body would address these issues. With such a system, sufferers of severe allergies would not need to carry an EpiPen® with them at all times, nor would they experience the risk of not being able to perform an injection during a severe attack.

### SUMMARY OF THE INVENTION

This invention resides in a system, device and method for automatically delivering medication for allergic reactions from within the body.

Apparatus, implanted subcutaneously or in muscle, fat, joint spaces, or body cavities of any type, includes a method of detecting an allergic and/or anaphylactic reaction. Such detection is carried out by monitoring the levels of biomarker molecules that indicate the occurrence of an allergic and/or anaphylactic reaction, such as: histamine, leukotrienes, prostaglandins, cytokines, tryptase, Fc-ε-RI complexes, anaphylatoxin C3a, chymase, carboxypeptidase A, platelet-activating factor², other mast cell degranulation byproducts, or other basophil activation compounds. A robust implementation of the invention further includes a system for dispensing an appropriate dosage of medication (such as epinephrine, antihistamines, or steroids) to mitigate the allergic/anaphylactic response. ² Sala Cunill, A., Cardona, V. Biomarkers of anaphylaxis, beyond tryptase. Current Opinion in Allergy and Clincal Immunology. Vol 15(4), August 2015, p 329-336. Doi: 10.1097/ACI.0000000000000184.

The simplicity and ease of use of this device has the potential to save many lives worldwide, as well as dramatically mitigate the risks of the current external methods of handling severe allergic reactions using external auto-injectors.

### BRIEF DESCRIPTION OF THE DRAWINGS

FIG. 1 is a block diagram of a preferred embodiment of the invention.

### DETAILED DESCRIPTION OF THE INVENTION

This invention includes a small (micro- or nano-scale) implantable device, operative to detect an allergic and/or anaphylactic reaction, and release stored medication (such as epinephrine, steroid, or antihistamine for example) for controlling the reaction. A method of detecting and measuring the levels of molecules indicates the occurrence of an allergic reaction, such as histamine, leukotrienes, prostaglandins, cytokines, and other inflammatory mediators and mast cell degranulation byproducts. The result of this detection signals the device to begin the controlled release of medication.

A minimally invasive surgical process is used to implant the device within, or below, the skin in either subcutaneous tissue, muscle, fat, joint spaces, or body cavities of any type. A controlled drug delivery system 104 within the device releases medication within the body, in an appropriate dosage, when elevated levels of chemical biomarker molecules of allergic or anaphylactic response are detected. In the preferred embodiment, the delivery system incorporates sufficient storage capacity for multiple dosages of the medication to be administered. Alternative embodiments may additionally include external and/or remote monitoring of the operations of the device's detection and/or release activities or medication levels.

### Device Operation Overview

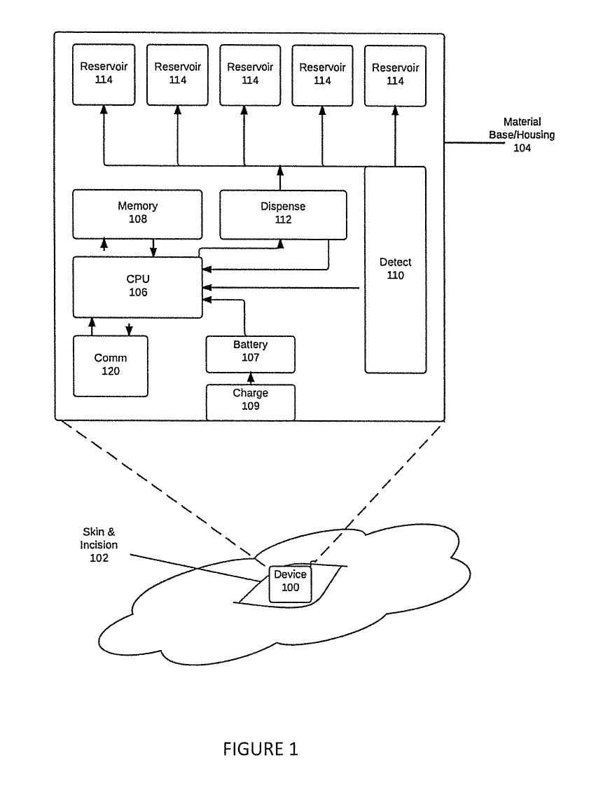

FIG. 1 provides an overview of the preferred embodiment. A physician, for example an ear, nose, and throat (ENT) or allergy specialist, could be responsible for installing and monitoring the implant 100 within or beneath the skin 102. The implant surgery would be a routine, outpatient procedure and may only require a small incision and local anesthetic.

The device is contained in a housing 104 constructed of biocompatible metal, plastic, polymer, hydrogel, or ceramic(s). The system includes a detector 110 described in further detail below that monitors specific biomarkers that are produced when an allergic or anaphylactic reaction occurs. Through the use of either an automatic chemically or electrochemically triggered pathway, or computation processor 106 (described below), the levels of one or more biomarkers (listed above) are used to initiate the release of medication through dispenser unit 112.

The system may be operated by a battery-powered, low-power programmed processor that controls all patient-specific functions. Memory 108 could store biomarker dectection specifics as well as information relating to medication type, appropriate dosage, etc. Battery 107 may be rechargeable, in which case it may be recharged inductively through charger 109 even while implanted.

The device would maintain a reserve of medication such that multiple dosages could be released without requiring device replacement. This is often called a “reservoir” device, referring to the fact that there is one or more reservoirs (114) that release medication over time. In this application, the medication would be delivered in predetermined safe and effective dosages corresponding to the detection outlined above. Certain levels of specific molecules could lead to varying dosages depending on the calculated “severity” of the reaction.

The device may have apparatus 120 and a method of communicating its need to be replenished with the necessary medication (epinephrine or other), at which point the patient would return to their physician for a refill.

### Detection of Biomarkers

As mentioned above, there are many molecules that can be used as indicators of the start of an allergic reaction. Histamine is one potential candidate, and some methods of detecting histamine are described below as illustrative examples of the type of processes that could facilitate the detection of any biomarker for an allergic or anaphylactic reaction. Histamine detection in living tissue appears to be a relatively new science. Below are three methods of detection that are currently being explored.

Minamiki et al. (2015)³ published an article presenting results on the use of an organic field effect transistor (OFET) to detect the presence of histamine in aqueous solutions, and output a voltage based on histamine presence or lack thereof. This research, originally motivated by a need for food freshness sensing in fish products, may be adapted through routine engineering modificationfor use in the inventive device. ³ Minamiki, T., Minami, T., Yokoyama, D., Fukuda, K., Kumaki, D., Tokito, S. 2015. Extended-gate organic field-effect transistor for the detection of histamine in water. Japanese Journal of Applied Pjysics, 54. http://dx.doi.org/10.7567/JJAP.54.04DK02

Alternatively, Cash and Clark (2012)⁴ published a study that discusses a successful method of using nanosensors to detect and measure histamine levels in mice. While this technology is early, it is a potential example of how micro-sensing technology could be used in conjunction with the delivery aspects of the device. ⁴ Cash, K., Clark, H. 2012. In Vivo Optical Histamine Nanosensors. Sensors, 12 (9). DOI: 10.3390/s12091192

Thirdly, Thredgold et al. (2015)⁵ have demonstrated that microchip electrophoresis with capacitively coupled contactless conductivity detection can be used to detect histamine levels in fish flesh. While this technology would need significant adaptation to be used practically within the body, it is another example of a different detection method applicable to the invention. ⁵ Thredgold, L, Ellis, A., Lenehan, C. 2015. Direct detection of histamine in fish flesh using microchip electrophoresis with capcaitively coupled contactless conductivity detection. Analytical Methods, 7. 1802. DOI: 10.1039/c4ay02866j

### Drug Delivery

There are many potential configurations for the drug delivery actuation mechanism. One possible model is an electrochemically activated device, which would leverage circuitry bridging attached nanosensors, to detect the presence of elevated histamine (or other chemical biomarkers) levels, and the actuation mechanism itself. As mentioned above, Cash and Clark have developed a method of a detecting histamine levels in vivo by using ionophores (transport molecules) to induce electrical charge rebalancing and a resulting change in optical properties. The choice of ionophore could be modified such that the effect would be cascading electrical signals instead of fluorescence, thereby providing a mechanism for the activation of drug delivery. For example, the current could initiate the breakdown or reconfiguration of a barrier material (biocompatible electrically responsive polymers are common and well researched⁶) thereby opening one of the device's reservoirs and making the medication available to the bloodstream. Alternatively, Minamiki et al.'s OFET system could be deployed in the circuitry directly. ⁶ Luo, R, Lim H, Lam, K Y. Modeling and analysis of pH-electric-stimuli-responsive hydrogels. J Biomater Sci Polym Ed. 2008;19(12):1597-610. doi: 10.1163/156856208786440532.

Another potential method of delivery is specially constructed biomaterials (e.g. hydrogels) that hold the drug of choice in an inert or inaccessible state until elevated histamine levels cause a change, either through molecular reconfiguration or degradation of an external protective layer. This technology could be incorporated into the concept from above. Additionally, the biomaterials themselves could be designed to fully degrade, exposing an core of medication (epinephrine or other), upon contact with sufficiently high concentrations of histamine; under this construction, varying levels of histamine protection could ensure that reserve is maintained for sufficiently severe reactions.

The NASA Biocapsule is one example of an implantable sensor and drug delivery device. Dr. David Loftus at the Space Biosciences division at NASA developed a system made of carbon nanotubes that are capable of detecting abnormalities in the body and delivering metered dosages of medication in response.⁷ One of Dr. Loftus' primary goals for the device is drug delivery for diabetic patients; this type of device would eliminate the need for patients to constantly measure blood sugar levels and carry medicine with them at all times. ⁷ http://gizmodo.com/5882725/the-miraculous-nasa-breakthrough-that-could-save-millions-of-lives

1. A system for controlling an allergic reation, comprising:
a detector operative to detect a physical or chemical biomarker molecule indicative of an allergic and/or anaphylactic reaction;
a reservoir containing a medication to mitigate an allergic or anaphylactic reaction;
a dispensing mechanism for dispensing an appropriate dosage of the medication in response to the detection of the reaction; and
wherein the detector, reservoir and dispensing mechanism are all contained in an implantable housing.

2. The system of claim 1, where the detector is operative to detect histamine, leukotrienes, prostaglandins, cytokines, tryptase, Fc-ε-RI complexes, anaphylatoxin C3a, chymase, carboxypeptidase A, platelet-activating factor, other mast cell degranulation byproducts, or other basophil activation compounds.

3. The system of claim 1, where the reservoir contains epinephrine, antihistamines, or steroids.

4. The system of claim 1, further including including apparatus for determining that a predetermined threshold of severity of a physiologic reaction has been met.

5. The system of claim 1, wherein the reservoir has sufficient capacity for storing multiple dosages to be administered independently.

6. The system of claim 1, wherein the housing is implanted within or below the skin in either the subcutaneous tissue, muscle, fat, joint spaces, or a body cavity.

7. The system of claim 1, further including circuitry for communincating status or operation external to the body in which the system is implanted.

8. The system of claim 7, wherein the circuitry communicates with an external monitoring device using radio frequency, Wi-Fi, Bluetooth, or other form of electromagnetic radiation.

9. The system of claim 8, wherein the external monitoring device is a computer or smartphone.

10. The system of claim 7, wherein the status information includes the medication level of the reservoir.
